(19) United States
(12) Patent Application Publication
(10) Pub. No.: US 2026/0321433 A1
(43) Pub. Date: Oct. 15, 2026

(54) HEALTHCARE PRE-SCREENING SYSTEM WITH MACHINE LEARNING

(71) Applicants: Alexander Brinley Gibson, Draper, UT (US); Spencer James Gibson, Henderson, NV (US)

(72) Inventors: Alexander Brinley Gibson, Draper, UT (US); Spencer James Gibson, Henderson, NV (US)

(21) Appl. No.: 19/643,583

(22) Filed: Apr. 9, 2026

Related U.S. Application Data
(60) Provisional application No. 63/787,585, filed on Apr. 11, 2025.

(51) Int. Cl. G16H 50/30; G06F 40/30; G16H 10/60; G16H 40/67; G16H 50/20; G16H 50/70

(52) U.S. Cl. CPC G16H 50/30; G06F 40/30; G16H 10/60; G16H 40/67; G16H 50/20; G16H 50/70

(57) ABSTRACT

A system includes at least one processing device and memory storing instructions that, when executed, cause the system to perform operations. The operations include receiving first data representing symptoms experienced by a user and second data representing a medical history, demographic information, or health metrics of the user. The operations include generating output embeddings using trained embedding models by processing the first and second data, wherein the output embeddings capture semantic relationships within the first and second data. The operations also include generating a user profile by comparing the output embeddings to predetermined embeddings. Additionally, the operations include generating probabilities for candidate conditions by evaluating the user profile against categories of medical information using a trained probabilistic classifier, and providing, via a user interface, one or more candidate conditions with associated probability scores. Additional methods and computer-readable media are also disclosed.

### CROSS-REFERENCE TO RELATED APPLICATIONS

This application claims priority to U.S. Application No. 63/787,585, titled “HEALTHCARE PRE-SCREENING SYSTEM WITH MACHINE LEARNING,” filed Apr. 11, 2025, which is hereby incorporated by reference in its entirety.

### FIELD OF INVENTION

The present disclosure relates to machine learning-based healthcare applications, and more particularly to an artificial intelligence-driven pre-screening system that uses embedding models and probabilistic inference to assess patient symptoms and provide personalized health risk evaluations.

### BACKGROUND

Healthcare pre-screening may involve assessing symptoms experienced by an individual to help determine whether medical attention may be warranted. Challenges can arise in this context when an individual's personal description of symptoms does not align with standardized medical terminology, potentially leading to missed or misinterpreted symptoms. For example, a user describing “burning sensations” may not recognize that such symptoms could correspond to “acid reflux” in medical terminology, which could lead to inaccurate conclusions. Additional challenges may include limited personalization based on a patient's medical history, demographic factors, or data from wearable devices. Further challenges may include a lack of mechanisms for providing quantified risk assessments tailored to individual users, or difficulty in the system presenting results in a user-friendly format that supports informed decision-making. Accordingly, improvements in healthcare pre-screening may be desirable.

### BRIEF DESCRIPTION OF THE DRAWINGS

To easily identify the discussion of any particular element or act, the most significant digit or digits in a reference number refer to the figure number in which that element is first introduced.

FIG. 1 illustrates a block diagram of a pre-screening system, according to aspects of the present disclosure.

FIG. 2 illustrates a block diagram of a data flow of a pre-screening system, according to an embodiment.

FIG. 3 illustrates an example embodiment of a user profile generated by the pre-screening system, according to aspects of the present disclosure.

FIG. 4 illustrates an example embodiment of a dataset of conditions generated by the pre-screening system, according to an embodiment.

FIG. 5 illustrates an example embodiment of a final output generated by the pre-screening system, according to aspects of the present disclosure.

FIG. 6 illustrates a flowchart for a routine for healthcare pre-screening using machine learning, according to an embodiment.

FIG. 7 illustrates a diagrammatic representation of a computing device, according to aspects of the present disclosure.

### DETAILED DESCRIPTION

When people feel unwell, they often struggle to describe their symptoms in the precise medical terms that doctors use. Someone might say they have “burning in their chest” without realizing this could indicate acid reflux, or describe “feeling dizzy” when a doctor would ask about vertigo. This mismatch between everyday language and medical terminology can make it difficult for individuals to accurately assess their health concerns and decide whether to seek medical care. The present disclosure addresses this challenge by providing a healthcare pre-screening system that understands what users mean, not just what they say, and combines this understanding with their personal health history to provide tailored, probability-based assessments of potential conditions.

To accomplish this, the system uses several key technologies working together. First, “embedding models” are software components that understand the meaning behind words, not just the words themselves. When a user says “burning in my chest,” the system recognizes this may relate to acid reflux even though those exact words were not used. Second, the system builds a “user profile” that summarizes the user's symptoms and health information by matching what the user describes to a database of medical knowledge (the “predetermined embeddings”). Third, a “probabilistic classifier” calculates how likely different conditions are based on all the evidence: the user's symptoms, their medical history, demographic factors, and patterns from broader health data. The result is a personalized list of potential conditions ranked by probability and severity, helping users make informed decisions about seeking care.

The present disclosure is generally directed to a healthcare pre-screening system with machine learning. The present disclosure describes a system comprising at least one processing device and memory storing instructions that, when executed by the at least one processing device, cause the system to perform operations. The operations include receiving first data representing one or more symptoms experienced by a user and second data representing one or more of a medical history of the user, demographic information, or a health metric corresponding to the user. The operations further include generating, using one or more trained embedding models, output embeddings by processing the first data and the second data, wherein the output embeddings capture semantic relationships within the first data and the second data. The operations also include generating a user profile by comparing the output embeddings to one or more predetermined embeddings using a vector similarity function, wherein each of the one or more predetermined embeddings represents a health data identifier. Additionally, the operations include generating a set of probabilities for candidate conditions by evaluating the user profile against one or more categories of medical information using a trained probabilistic classifier, and providing, via a user interface, a ranked list of candidate conditions, wherein each candidate condition is associated with a probability score from the set of probabilities and a severity level.

Aspects and embodiments of the present disclosure provide a pre-screening system that uses at least one embedding model to capture the semantic meanings of reported user symptoms and the semantic meanings of symptoms listed in symptom libraries and compare the different output embeddings. This approach allows users to describe their symptoms using different keywords or phrases than are used in the library and still be matched to corresponding symptoms in the library. This may reduce the risk that a user will not find a symptom they are experiencing because they described the symptom differently than the symptom library.

Some disclosed embodiments may also incorporate medical history and demographic data into the models. By accounting for such factors, disclosed embodiments may provide assessments reflecting an elevated likelihood that a particular disease or symptom applies to a given individual. Users may thereby gain insight into personal risk factors when deciding whether to seek medical attention.

Condition probabilities based on quantified health risks may be computed and presented in a user-friendly format. A Bayesian inference model may integrate symptom data with medical history and population-level datasets to calculate these probabilities. The resulting output may include a ranked list of conditions, each associated with a probability score and severity level, with more severe conditions and higher probability scores presented first to support informed decision-making. Because the probabilities may reflect not only current inputs but also patterns derived from broader population and wearable data, the system can generate condition likelihoods tailored to the individual, potentially improving the clarity and clinical relevance of its recommendations.

Some embodiments support continuous refinement through longitudinal tracking, storing and reusing health-related data collected across multiple interactions to improve the accuracy of future assessments. By updating evaluations based on previous interactions, the system may enable progressively more accurate risk assessments over time.

As used herein, a “health data identifier” refers to any code, label, or standardized identifier used to represent a medical concept, and includes medical codes such as those from the International Classification of Diseases (ICD), the Diagnostic and Statistical Manual of Mental Disorders (DSM-5), the Current Procedural Terminology (CPT), the Healthcare Common Procedure Coding System (HCPCS), or similar diagnostic or procedural coding systems. As used herein, the term “trained probabilistic classifier” refers to a machine learning model or statistical model that has been trained to generate probability estimates for candidate outcomes based on input data. A Bayesian inference model is a type of trained probabilistic classifier. As used herein, “first data” refers to data representing one or more symptoms experienced by a user, and may be received via text input or voice-to-text conversion. As used herein, “second data” and “user health data” are used interchangeably to refer to data representing one or more of a medical history of the user, demographic information, or a health metric corresponding to the user. As used herein, a “symptom library” refers to a structured collection of medical symptoms, conditions, or health concepts, and may include or be derived from standardized medical coding systems such as the International Classification of Diseases (ICD), the Diagnostic and Statistical Manual of Mental Disorders (DSM-5), the Current Procedural Terminology (CPT), the Healthcare Common Procedure Coding System (HCPCS), or similar diagnostic or procedural coding systems. As used herein, a “compact embedding model” refers to an embedding model that may be constructed with a reduced number of parameters and smaller architecture sizes to enable efficient on-device execution, and may include, but is not limited to, models such as Distilled Bidirectional Encoder Representations from Transformers (DistilBERT), A Lite Bidirectional Encoder Representations from Transformers (ALBERT), or Tiny Bidirectional Encoder Representations from Transformers (TinyBERT), or other embedding models that have been optimized using quantization techniques, knowledge distillation, or other model compression methods.

When executed, the instructions may cause the system to receive first data representing one or more symptoms experienced by a user, along with second data representing medical history, demographic information, or health metrics. A user interface may allow entry of this input, and the system may prompt the user to answer questions about symptoms, medical history, demographics, or health metrics.

Execution of the instructions may also cause the system to generate output embeddings by processing the first data and the second data through one or more trained embedding models. These output embeddings may be vectors whose values capture semantic relationships, including the semantic meaning of portions of the input data. To generate a user profile, the system may compare the output embeddings to predetermined embeddings representing health data identifiers. The predetermined embeddings may have been generated by embedding identifiers from a symptom library or standardized medical coding system, such as the International Classification of Diseases (ICD), the Diagnostic and Statistical Manual of Mental Disorders (DSM-5), the Current Procedural Terminology (CPT), or the Healthcare Common Procedure Coding System (HCPCS). The resulting user profile may list symptoms the user is likely experiencing based on the comparison, along with medical history factors, demographic information, or health metrics related to those symptoms. Such additional factors can be included even when their output embeddings do not match a predetermined embedding representing a health data identifier.

The system may further generate a set of probabilities by evaluating the user profile against one or more datasets, each corresponding to a different category of medical information. These datasets may include wearable and biometric data, medical history and records, demographic data, community health trends, and integrated symptom-health risk probability data. A trained probabilistic classifier, such as a Bayesian inference model, may generate the probabilities, which represent the likelihood that a user has a particular condition or disease. Through the user interface, the system may provide a final output listing potential conditions with corresponding probabilities and severity levels (e.g., severe or moderate) to help the user decide whether to seek medical attention. The severity may be computed by the trained probabilistic classifier using the datasets. The final output may also include suggested actions; for example, if a life-threatening condition such as a heart attack or stroke appears likely, the output may recommend visiting an emergency room or calling an ambulance, whereas a less urgent but probable condition such as a cold or migraine may prompt a recommendation to schedule an appointment with a family doctor.

The healthcare pre-screening system of the present disclosure provides several technical advantages. First, the use of trained embedding models to capture semantic relationships within user-reported symptoms eliminates the reliance on exact keyword matching that characterizes conventional symptom-checker applications. This semantic approach allows users to describe symptoms using natural language and personal terminology while still achieving accurate matching to standardized medical codes. As a result, the risk that relevant symptoms are overlooked due to vocabulary mismatches between user descriptions and symptom library entries is reduced. Second, the trained probabilistic classifier integrates multiple categories of medical information, including wearable and biometric data, medical history records, demographic data, community health trends, and integrated symptom-health risk probability data. This integration enables personalized probability assessments that account for individual risk factors rather than providing generic recommendations. Third, the system generates quantified probability scores for candidate conditions rather than binary or categorical outputs. These differentiated risk assessments provide users with information that supports more informed decision-making about whether and when to seek medical attention. Fourth, compact embedding model architectures and quantization techniques enable efficient on-device execution on mobile platforms. This approach supports user privacy by storing sensitive health information locally and provides responsive operation in connectivity-limited settings. Fifth, the local user profile database enables longitudinal tracking and continuous refinement of assessments based on prior interactions. This capability allows the system to achieve progressively more accurate risk assessments over time without requiring full model retraining. Collectively, these technical advantages contribute to a healthcare pre-screening solution that provides semantically-aware symptom matching, personalized probabilistic assessments, and efficient on-device operation.

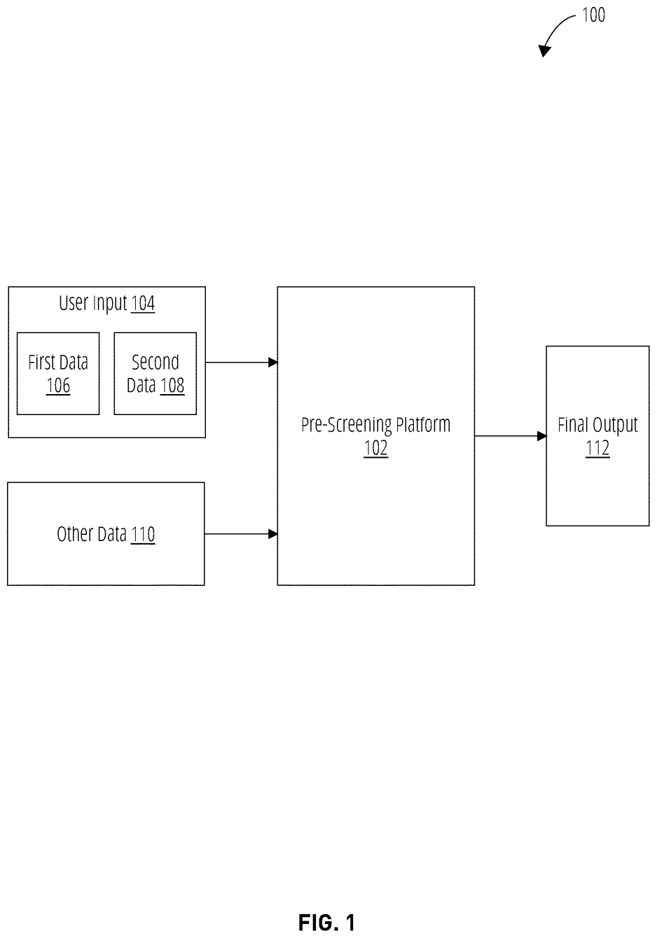

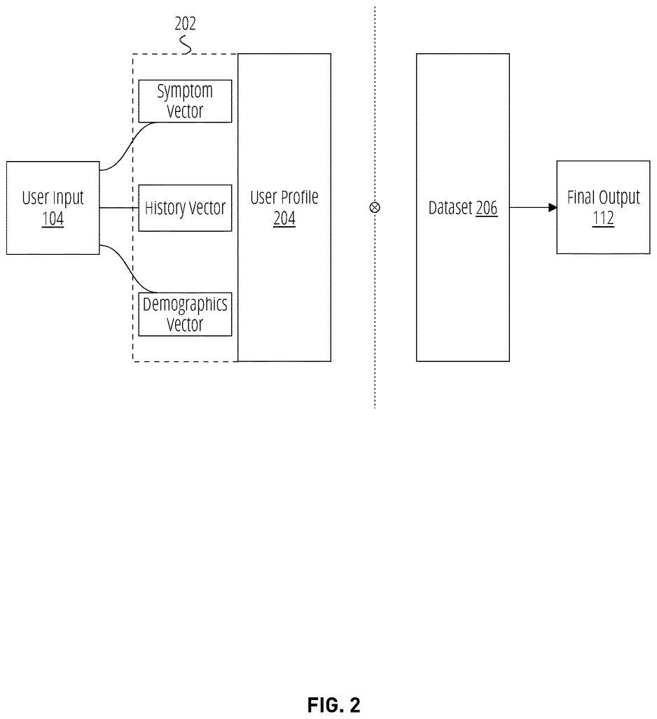

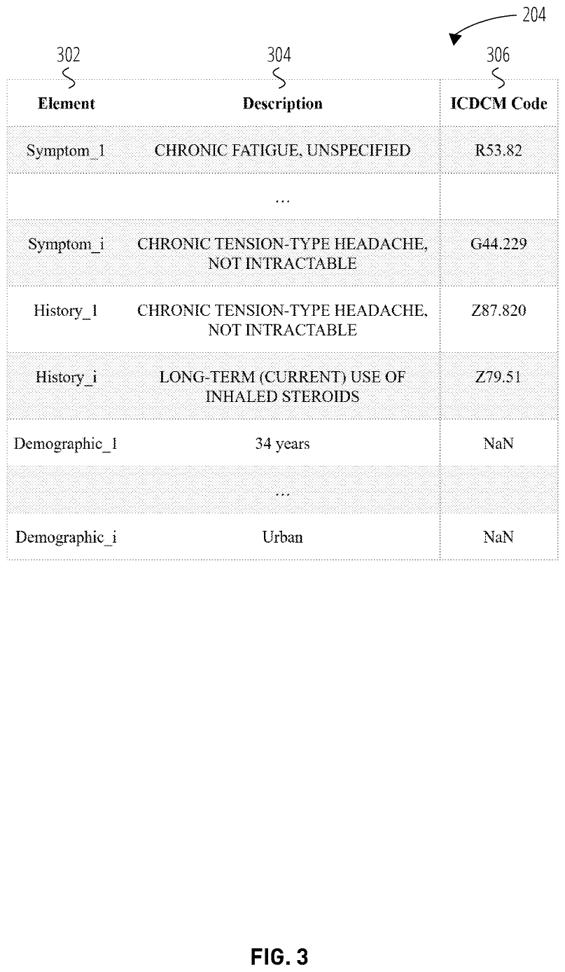

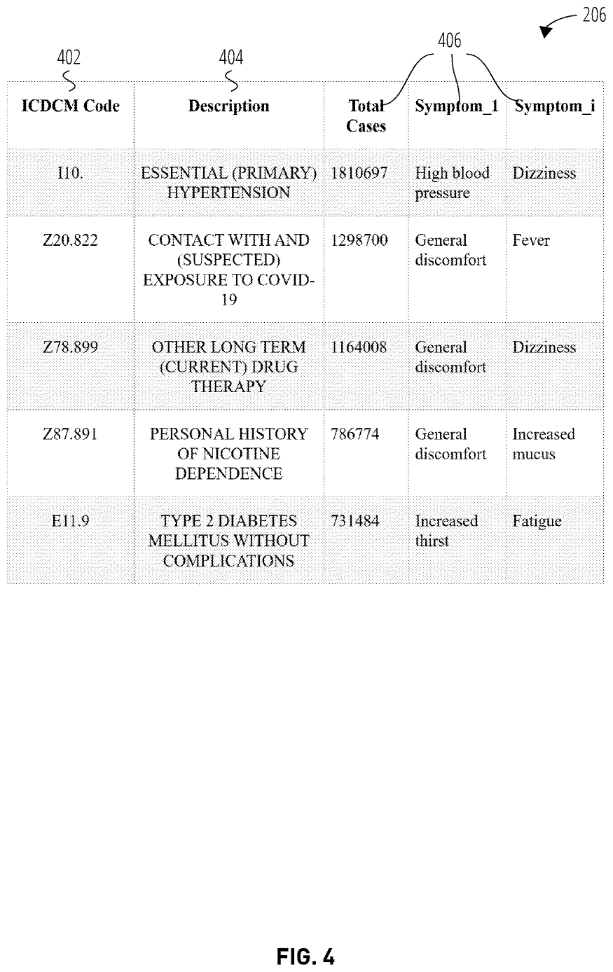

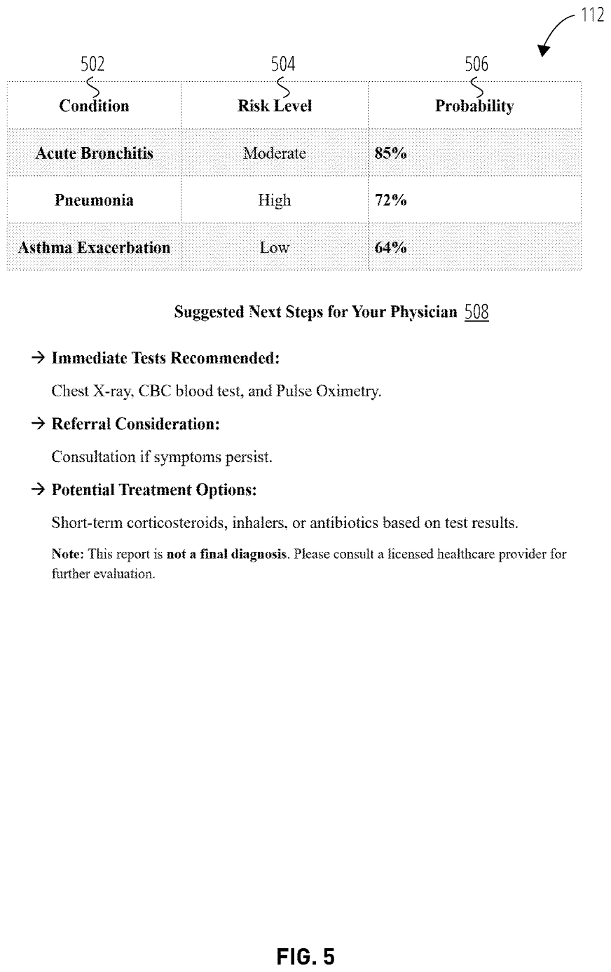

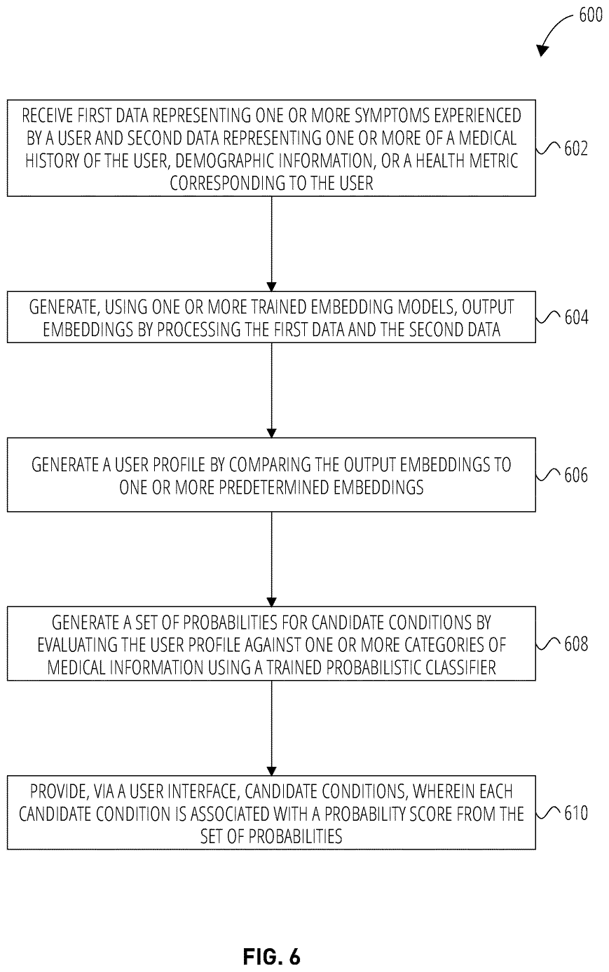

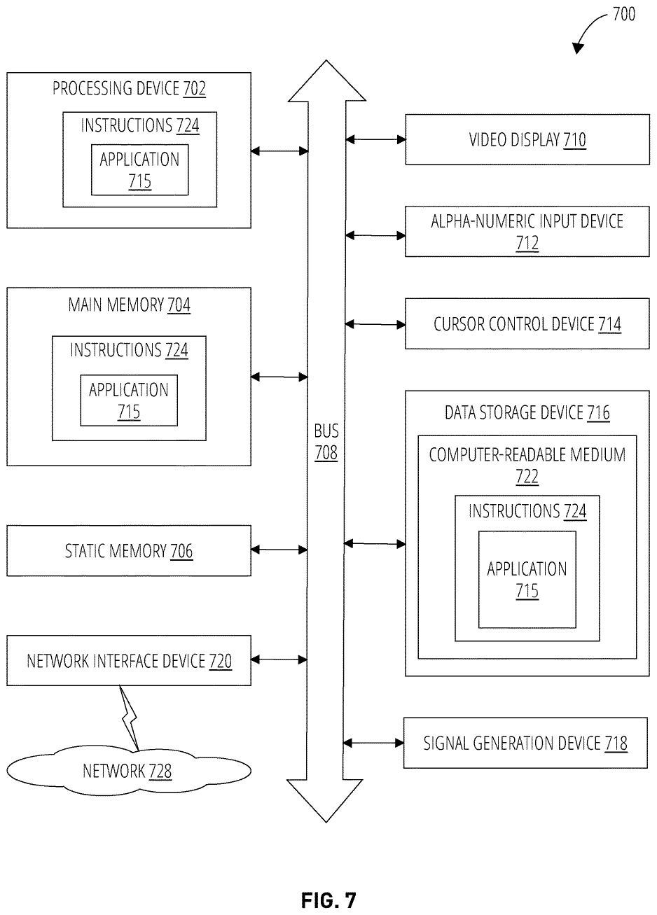

The following description refers to the accompanying drawings, which illustrate various aspects of the pre-screening system and its operation. FIG. 1 illustrates a block diagram of the pre-screening system architecture, showing the relationship between user input, other data sources, the pre-screening platform, and the final output. FIG. 2 depicts the data flow through the pre-screening system, illustrating how user input is processed through embedding models to generate output embeddings, which are then used to create a user profile and ultimately produce a final output via the trained probabilistic classifier. FIGS. 3 and 4 provide example embodiments of intermediate data structures generated during system operation, with FIG. 3 showing a user profile containing matched symptoms and factors with their corresponding descriptions and health data identifiers, and FIG. 4 showing a dataset of candidate conditions with associated diagnostic codes, descriptions, and statistical data used in probability generation. FIG. 5 illustrates an example embodiment of the final output presented to the user, including a ranked list of conditions with associated severity levels, probability scores, and suggested next steps. FIG. 6 illustrates a flowchart for a routine for healthcare pre-screening using machine learning. FIG. 7 depicts a diagrammatic representation of a computing device that may be used to implement various embodiments of the pre-screening system.

FIG. 1 illustrates a block diagram of a pre-screening system 100 according to some embodiments. The pre-screening system 100 may include a pre-screening platform 102. The pre-screening platform 102, as will be described in more detail throughout the disclosure, may include a plurality of models that are used to generate a final output 112. The final output 112 may include probabilities representing how likely a user is to be experiencing a particular condition, symptom, or disease based on the user's user input 104 and other data 110 that are input into the pre-screening platform 102. The pre-screening platform 102 may be a cross-platform system that can operate as a mobile application, a web-based application, or as a cloud-based application. In some embodiments, the system may be implemented on a mobile device, such as a smartphone or tablet.

The pre-screening platform 102 may include one or more embedding models. In some embodiments, one or more of the embedding models are models such as a Word2Vec model or a FastText model that may implement a neural network architecture. In various embodiments, one or more of the embedding models are models such as a Bidirectional Encoder Representations from Transformers (BERT) model or a Generative Pre-Trained Transformer (GPT) model that implements a transformer model. In some embodiments, one or more of the embedding models may be a modified large language model (LLM). Many LLMs include an internal embedding model as one stage of the LLM where the internal embedding model generates embeddings that are passed through additional stages in the LLM to produce an overall output. A developer or implementer may modify an LLM by programming it to provide the generated embeddings produced by its internal embedding model as an output, instead of passing the generated embeddings to the additional stages of the LLM. The modified LLM may be obtained by programming a GPT series LLM, a BERT LLM, a Large Language Model Meta AI (LLaMA) model, a Pathways Language Model (PaLM), or the like to act as an embedding model for the pre-screening platform 102.

More than one embedding model may be used in pre-screening platform 102 to leverage the advantages of particular embedding models to obtain more accurate or nuanced embeddings of the user input 104 when compared with generalized embedding models. For example, one embedding model may be a modified LLM that was pre-trained to embed and capture the context for symptom information while another embedding model may be a modified LLM that was pre-trained to embed and capture the context for medical history information. In other cases, one or more modified LLMs used in the pre-screening platform 102 may have been pre-trained to embed and capture the contexts for different conditions, e.g., including one modified LLM trained to embed input related to heart diseases, another LLM trained to embed input related to types of cancers, and additional LLMs for additional categories of diseases. In some embodiments, one or more of the embedding models may be an embedding model that is trained to embed and capture the context for symptom information, medical history information, health metrics, or the like, as previously described with the examples of modified LLMs. In some embodiments, the system may employ a routing mechanism to determine which of the multiple embedding models processes different types of input data. The routing logic may be based on input classification, keyword detection, or learned routing functions. The routing mechanism may direct different types of input data to specialized embedding models pre-trained for specific data categories, which may improve embedding quality. In some embodiments, the routing mechanism is not required when a single general-purpose embedding model is employed.

In some embodiments, the pre-screening platform 102 may handle multi-language input for symptom descriptions. The one or more embedding models may include multilingual embedding models, such as multilingual Bidirectional Encoder Representations from Transformers (multilingual BERT), that are pre-trained on text corpora spanning multiple languages to generate semantically meaningful embeddings regardless of the input language. In other embodiments, the system may employ language-specific embedding models paired with translation preprocessing, where user input in a non-English language is translated to a target language before embedding generation. Cross-lingual transfer learning techniques may also be applied, enabling embedding models trained primarily on one language to generalize semantic understanding to other languages. These approaches may support non-English semantic understanding, allowing users to describe symptoms in their native language while maintaining accurate semantic matching against predetermined medical code embeddings.

In some embodiments, the embedding models may alternatively be implemented using recurrent neural network architectures such as Long Short-Term Memory (LSTM) or Gated Recurrent Unit (GRU) networks that process sequential symptom descriptions to generate fixed-length embedding vectors, or using convolutional neural networks (CNNs) applied to character-level or word-level representations of medical text. The similarity comparison between output embeddings and predetermined medical code embeddings may alternatively employ learned metric functions using Siamese networks (neural networks that compare two inputs by processing them through identical subnetworks) or triplet networks (neural networks that learn relationships among anchor, positive, and negative examples) trained to directly optimize embedding distances for medical concept matching tasks. The user profile generation may alternatively utilize graph-based representations where symptoms, medical history factors, and demographic attributes are represented as nodes with weighted edges encoding semantic relationships, enabling graph neural network processing to generate user embeddings.

The embedding models or modified LLMs may be pre-trained using a full or partial fine-tuning method where the entire model (full) or parts of the model (partial) are trained on a labeled dataset, e.g., a prepared dataset with question-answer (QA) pairs, classification labels, or the like. In some embodiments, the embedding models or modified LLMs may be pre-trained using an instruction tuning or supervised fine-tuning (SFT) method. The embedding models or modified LLMs may be fine-tuned or pre-trained for similarity or classification by training the models with prepared positive pairs (e.g., texts labeled as similar) and negative pairs (e.g., texts labeled as dissimilar) and use contrastive learning (training that teaches the model to distinguish similar from dissimilar examples) or triplet loss (a training objective using anchor, positive, and negative example comparisons) to optimize the output embeddings of the model so that similar pairs have similar generated output embeddings. Validation methods to ensure accuracy of semantic matching between user symptom descriptions and predetermined medical code embeddings may include expert annotation comparison, cross-validation against clinical datasets, and evaluation metrics such as precision, recall, and F1 scores on held-out test sets.

In some embodiments, the pre-screening platform 102 includes one or more embedding models that are designed to be compact and computationally efficient. These models may be constructed with a reduced number of parameters (e.g., under 100 million) and use smaller architecture sizes, such as Distilled Bidirectional Encoder Representations from Transformers (DistilBERT) (66M parameters), A Lite Bidirectional Encoder Representations from Transformers (ALBERT) (12-18M parameters), or Tiny Bidirectional Encoder Representations from Transformers (TinyBERT) (4-15M parameters). In some embodiments, a threshold number of parameters may be defined based on deployment constraints, such as a maximum parameter count (e.g., 100 million parameters), a maximum model size (e.g., under 1 GB), or computational requirements for real-time inference on mobile device processors. A compact embedding model may comprise a transformer-based architecture with fewer parameters than the threshold number of parameters. These compact architectures reduce computational overhead while retaining the ability to generate semantically meaningful embeddings. To further decrease memory footprint and processing demands, the models may apply quantization techniques such as 8-bit or 4-bit integer quantization using post-training static quantization (reducing numerical precision after training is complete) or dynamic quantization (adjusting precision during inference), which allows the models to run efficiently on mobile central processing units (CPUs) without dedicated AI hardware. Additionally, knowledge distillation may be employed to train these compact models by transferring learned patterns from a larger model into a smaller model (e.g., GPT-40 or BERT-Large), with the compact model acting as a student model. In some embodiments, the compact on-device model may alternatively or additionally be implemented using neural network pruning techniques that remove low-magnitude weights, structured pruning that eliminates channels or layers, or lottery ticket hypothesis methods that identify sparse subnetworks achieving comparable performance to full models. In these cases, the student model learns to approximate the teacher's embedding outputs or prediction distributions by minimizing a loss function such as Kullback-Leibler divergence (a measure of how different the student's outputs are from the teacher's outputs) between teacher and student outputs during training. These optimizations enable local operation of the models on personal devices such as smartphones and tablets, with total storage constraints typically under 1 GB. This offline functionality supports user privacy and responsiveness, particularly in resource-constrained or connectivity-limited settings. In some embodiments, the one or more trained embedding models may execute locally on a processor of the mobile device using TensorFlow Lite, Core ML, or equivalent on-device machine learning framework (collectively, an on-device machine learning framework).

The pre-screening platform 102 may also include a trained probabilistic classifier. The trained probabilistic classifier may be, for example, a Bayesian inference model, and may generate a set of probabilities that represent the probability that a user is experiencing a particular symptom, condition, or disease. To generate the probabilities, the trained probabilistic classifier may include an embedding model to make an initial prediction of the conditions, symptoms, or diseases a user may be experiencing (as further described with FIG. 2). For example, the trained probabilistic classifier can compare received data, such as embedding(s) of upstream inference models, to predefined datasets by estimating the probability that the received data belongs to each predefined class or group, given prior knowledge and observed evidence. This process may begin with initial estimations that reflect the likelihood of the new data matching each predefined embedding or group. As the model observes the received data, a likelihood function may be used to assess how probable the received data is under each hypothesis (e.g., similarity to a predefined embedding set). Bayes' theorem (a mathematical formula for updating probability estimates based on new evidence) can then be applied to update the initial probability estimates based on this new evidence, producing revised probabilities for each hypothesis that reflect both the prior knowledge and the observed data. This approach can enable the trained probabilistic classifier to quantify uncertainty and make probabilistic comparisons (i.e., rather than binary decisions). The trained probabilistic classifier may generate the set of probabilities by updating the initial prediction based on datasets provided in the other data 110 (described in more detail below). The trained probabilistic classifier allows existing information, e.g., the datasets of the other data 110, to be incorporated into the probability calculation for a particular hypothesis, e.g., the hypothesis that the user is experiencing a particular condition, symptom, or disease, and allows for full probability distributions to be generated for parameters to quantify the probability of the hypothesis being true. The trained probabilistic classifier may calculate probability estimates for candidate conditions based on the user profile, and the set of probabilities may be generated based on the probability estimates. For example, if a patient has symptoms S, past clinical and condition history H, and a candidate condition they may be suffering from C, the probability that the patient may show their current symptoms given their past history if they had the candidate condition may be modeled as shown below:

$$Pr ( S \vert C , H ) = \frac{Pr ( C \vert S , H ) Pr ( S \vert H )}{Pr ( C \vert H )}$$

In the above equation, the vertical bars “|” denote conditional probabilities. For example, Pr(C|S,H) is read as “the probability of condition C given symptoms S and history H.” This expresses how likely condition C is to be experienced by the user, assuming the user is experiencing symptoms S and has a medical history H. The other probability terms in the above equations are defined as follows: Pr(C|S,H) is the posterior probability that the user has condition C given their symptoms S and medical history H. This is what the system aims to estimate. Pr(S|C,H) is the likelihood, e.g., the probability of observing the symptoms S assuming the user has condition C and history H. Pr(C|H) is the prior probability of condition C given the medical history H, before considering current symptoms. Pr(S|H) is the marginal probability of symptoms S given history H, used for normalization.

Relevant conditions are those which achieve the largest values for the above probability. The largest values for the above probability may be calculated using:

$${C}^{*} = arg {max}_{c} Pr ( S \vert C , H ) = arg {max}_{c} ( \frac{Pr ( C \vert S , H ) Pr ( S \vert H )}{Pr ( C \vert H )} ) = arg {max}_{c} \frac{Pr ( C \vert S , H )}{Pr ( C \vert H )}$$

In the above equation, the equality term on the far right removes the prior Pr(S|H) because it has no dependence on the condition. The approach can be used to provide the top N conditions a user is likely to be experiencing. The unknown quantity Pr(C|S,H) may be estimated with a function ƒ_{Θ,D} where the subscripts show parametrization with Θ and dependence on a dataset D, e.g., ƒ_{Θ,D}(c)=\hat{P}r(c|S, H). The term Pr(C|H) may be estimated with a parametrized function g_{φ,D}, for some parameter set φ and dataset D′. For a patient without a provided medical history, the terms in the equality chain above simplify to population probabilities.

In some embodiments, the trained probabilistic classifier may additionally or alternatively employ probabilistic frameworks including Markov chain Monte Carlo (MCMC) sampling methods (techniques that generate samples from probability distributions through iterative random sampling), variational inference approximations, or ensemble methods combining multiple probabilistic classifiers such as naive Bayes, logistic regression, and random forests to generate condition probability estimates. In some embodiments, the system may alternatively generate probability sets using frequentist statistical methods including maximum likelihood estimation with confidence intervals, or discriminative models such as gradient boosting classifiers or support vector machines calibrated to output probability estimates.

The pre-screening platform 102 may include a user interface. The user interface may be a graphical user interface (GUI), a command line interface (CLI), a menu-driven interface, a touchscreen interface, a conversational interface, a voice user interface (VUI), a natural language interface, or the like. The user interface may be programmed to allow a user to enter user input 104 into the user interface through peripheral devices, e.g., wired keyboards, wireless keyboards, microphones, computer mice, touch screen or the like. The user interface may be programmed to obtain user input 104 corresponding to displayed prompts or questions for the user. In some embodiments, these prompts or questions may be personalized to the user based on the user's medical history or related demographic information. The user input 104 may be provided by the user in any suitable form, such as text that a user enters into the user interface, a text file uploaded to the user interface, an audio file that is to be converted to text by the user interface where the user interface includes speech-to-text capabilities, or the like. The first data may be received via text input or voice-to-text conversion, and may be provided in any of multiple languages supported by the one or more embedding models, including through multilingual embedding models or translation preprocessing.

The user input 104 may include first data 106 that represents one or more symptoms experienced by a user, such as whether the user is feeling pain, where the user is feeling pain, if the user is experiencing fatigue, descriptions of what kind of pain or discomfort the user is feeling, or the like. The user input 104 may also include second data 108 that represents additional medical information related to the user. For example, the additional medical information may represent the medical history of the user and include information about whether the individual is currently taking any medications, family medical history, prior hospitalizations, prior diagnoses, or the like. Additionally, the additional medical information may represent demographic information that may include age, ethnicity, gender, current environmental conditions (such as urban, rural, etc.), employment status, or the like.

The pre-screening system 100 may also include other data 110 that is input into the pre-screening platform 102. The other data 110 may be multiple sets of datasets. Each of the multiple sets of datasets may be one of wearable and/or biometric data, a medical history and records set of data, demographic data, community health trends data, and integrated symptom-health risk probability data. The wearable and biometric data set may contain data obtained from wearable devices worn by the user that can track health metrics of the user, such as the user's heart rate, activity levels, sleep patterns, steps taken, calories burned, body temperature, electrocardiogram (ECG) signals, muscle activity, blood pressure, blood glucose levels, or the like. The wearable devices may include smartwatches, fitness trackers, smart rings, wearable ECG monitors, smart clothing, wearable blood pressure monitors, wearable continuous glucose monitors (CGMs), or the like. The wearable device data may be accessed through platform-specific health data application programming interfaces (APIs) or software development kits (SDKs) provided by mobile operating systems or device manufacturers. Data preprocessing steps applied to wearable device data before integration into the trained probabilistic classifier may include normalization, outlier detection, temporal aggregation, missing value imputation, and conversion to standardized health metric representations. The wearable and biometric data set may also include data obtained from a user's medical devices which may include pacemakers, defibrillators, hearing aids, remote blood pressure monitors, and implanted CGMs.

The medical history records set of data may include data previously obtained from the user that may include digitized medical records, electronic records requested or received from medical offices, medical records or medical information of family members, previous diagnoses, prescriptions, treatments received, vaccination records, or the like. The demographic data may include data related to statistics or trends of symptoms, diseases, or conditions experienced by different groups of the population separated into categories based on factors like age, race, gender, ethnicity or the like. For example, the demographic data may include statistics representing the prevalence of a particular type of heart disease for males over the age of fifty. The community health trends data may include data that represents health trends for the community of the user, such as spikes in the number of reports of a particular illness in nearby medical facilities. The integrated symptom-health risk probability data may include data that represents the likelihood of an individual having a disease based on the symptoms an individual is experiencing and risk factors such as genetic predisposition, lifestyle choices, and environmental exposures.

The pre-screening platform 102 may generate a final output 112 to be displayed to the user through the user interface. The final output 112 may display a predetermined number (such as 10) of the conditions, symptoms, or diseases the user is more likely to be experiencing. The final output 112 may include probabilities (described in more detail with FIG. 2) associated with each listed condition, symptom, or disease; a severity (described in more detail with FIG. 5) associated with each condition, symptom, or disease; and suggested actions (described in more detail with FIG. 5) the user may take to address the conditions, symptoms, or diseases the user may be experiencing.

FIG. 2 illustrates a block diagram of a data flow 200 of the pre-screening system 100 of FIG. 1, according to some embodiments. The data flow 200 may consist of a series of stages. In a first stage, pre-screening system 100 may provide user input 104 obtained from the user at the user interface to the one or more embedding models (not depicted). The one or more embedding models may embed the user input 104 into different vectors or output embeddings 202. The different vectors or output embeddings 202 may represent the semantic meaning or captured context of different portions of the user input 104. For example, portions of the first data 106 may be embedded using an embedding model pretrained on symptom information to obtain symptom vectors (numerical representations that capture the meaning or context of symptoms described in a given text sample, e.g., the first data 106). The different vectors or output embeddings 202 may be classified into different categories of vectors such as symptom vectors, history vectors, and demographic vectors. As previously described, the symptom vectors may capture the semantic meaning of portions of the first data 106 that correspond to descriptions of the user's symptoms. Similarly, the history vectors may capture the semantic meaning of portions of the second data 108 that correspond to descriptions of the user's medical history, family medical history, past diagnoses, or the like, and the demographic vectors may capture the semantic meaning of portions of the second data 108 that correspond to descriptions of the user's demographics, such as the user's age, race, gender, ethnicity, or the like.

In a second stage of data flow 200, the symptom vectors of the output embeddings 202 may be provided to the trained probabilistic classifier to make an initial prediction of the conditions, symptoms, or diseases the user is experiencing. The initial prediction may include using one of the one or more embedding models to determine the similarity between the output embeddings 202 and predetermined embeddings of symptoms in a symptom library or a standardized medical coding system, such as the ICD, the DSM-5, the CPT, the HCPCS, or the like. The embedding model used to determine the similarity between the output embeddings 202 and the predetermined embeddings may have been used to generate the predetermined embeddings of the symptoms. The embedding model may be programmed to determine the similarity between the symptom vectors of the output embeddings 202 and the predetermined embeddings by using a vector similarity function such as cosine similarity, dot product similarity, mean square error (MSE) or mean square error distance, Euclidean distance (a measure of straight-line distance between points in vector space), Jaccard similarity (a measure of overlap between sets), or Hamming distance (a count of differing positions between vectors). The results of the comparison of the output embeddings 202 and the predetermined embeddings may be used to generate a user profile 204, such as the example embodiment of user profile 204 depicted in FIG. 3. As shown in FIG. 3, the user profile 204 may include an initial set of predictions of what symptoms a user may be experiencing by listing what symptoms (and their corresponding embeddings) were entered by the user that had a similar context or meaning compared to the symptoms of the symptom library or standardized medical coding system (and their corresponding predetermined embeddings).

In some embodiments, when a user's symptom description generates output embeddings that do not meet a similarity threshold with any predetermined embeddings in the symptom library, the system may handle the unmatched input in one or more ways. For example, the system may flag the input for manual review by a healthcare professional or system administrator. Alternatively, the system may request clarification from the user through the user interface, prompting the user to provide additional details or rephrase the symptom description. In some cases, the system may apply a fallback matching strategy using broader semantic categories, where the output embeddings are compared against higher-level or more general medical concept embeddings rather than specific symptom codes. These approaches may help ensure that user inputs that do not closely match predetermined embeddings are still processed in a meaningful way, reducing the likelihood that relevant symptoms are overlooked during user profile generation.

The user profile 204 may also list factors, such as factors corresponding to the history vectors or the demographic vectors, that were not similar to a symptom of the symptom library but were similar to predetermined embeddings for historical factors and demographic factors. Examples of the symptoms and the factors entered by the user in the user input 104 that may be listed in the user profile 204 are represented as elements 302 in FIG. 3. The user profile 204 may also include the corresponding descriptions 304 and health data identifiers corresponding to the symptoms or factors that were similar between the output embeddings and the predetermined embeddings.

Returning to the description of FIG. 2, in a third stage of data flow 200, the trained probabilistic classifier may update the predictions of the listed entries of the user profile 204 using the datasets provided with the other data 110 to generate sets of probabilities that represent the likelihood that a user is experiencing a particular condition, symptom, or disease. When different datasets in the other data 110 provide contradictory evidence for the same condition, the system may resolve conflicting probability assessments through weighted averaging based on data source reliability, recency weighting, or hierarchical Bayesian aggregation that accounts for uncertainty in each data source. The trained probabilistic classifier may generate the sets of probabilities by computing the probability that the user is experiencing each candidate condition in the dataset 206, given the combination of symptoms, history, and demographic vectors extracted from the user profile 204, using a relationship between the different combinations such as:

Pr(C|S,H)∝Pr(S|C,H)·Pr(C|H)

The trained probabilistic classifier (e.g., the Bayesian inference model) may use the set of probabilities to generate a dataset 206, such as the example embodiment of the dataset 206 depicted in FIG. 4, that contains the conditions, symptoms, or diseases the user is more likely to have or to be experiencing along with associated statistics used to generate the set of probabilities. The dataset 206 may comprise an ordered or ranked collection of entries, where entries are ordered by their associated probability scores. As depicted in FIG. 4, each entry in the dataset 206 may correspond to a particular condition, symptom, or disease with a corresponding diagnostic code 402 and a corresponding description 404. The dataset 206 may also include data associated with the condition 406. As depicted in FIG. 4, the data associated with condition 406 may include statistics such as the total reported cases of the condition, symptom, or disease, and may also include the more common symptoms associated with the condition (shown as symptom 1 and symptom i in FIG. 4). The size and/or length of the dataset 206 may be determined by a predetermined threshold. For example, the size and/or length of the dataset 206 may be limited to 10 entries when the predetermined threshold is set to 10 so that the entries in the dataset 206 represent the 10 conditions, symptoms, or diseases the user is more likely to have or experience based on the user input 104 and the other data 110. While an example threshold was set at 10 entries, one of ordinary skill in the art will recognize that more than 10 entries or fewer than 10 entries may be included in the dataset without departing from the scope of the present disclosure.

Returning to the description of FIG. 2, in a fourth stage of data flow 200, the pre-screening system 100 generates the final output 112 (example embodiment depicted in FIG. 5) using the dataset 206 and the set of probabilities generated by the trained probabilistic classifier. While four stages of the data flow 200 are discussed, one of ordinary skill in the art will recognize that additional stages may be added without departing from the scope of the present disclosure. For example, additional embedding or normalization (adjusting data to a common scale) stages may be added between the first stage of data flow 200 and the second stage of data flow 200 to generate the output embeddings 202. Additionally, each embedding model of the one or more embedding models may constitute a stage of the data flow 200.

FIG. 5 represents an example embodiment of the final output 112 generated by the pre-screening platform 102. The final output 112 may list conditions 502 the user is likely to have or experience. In some embodiments, the final output 112 may also list diseases or symptoms the user is likely to have or experience. The final output 112 may also include a severity level 504 associated with each condition, symptom, or disease listed. The severity level 504 may be a ranking, such as low, moderate, and severe, where a severe ranking indicates that the condition, symptom, or disease, if experienced by the user and left untreated, could lead to death or permanent injury and a low ranking indicates that the condition, symptom, or disease, if experienced by the user and left untreated, poses low risk to the health of the user, such as resulting in unnecessary discomfort to the user. The system may determine the severity level by referencing stored severity mappings associated with each ICD code, or alternatively, calculated dynamically using the trained probabilistic classifier based on factors such as historical case outcomes, population-level mortality and morbidity (illness or disease rates) data, and the user's individual risk profile. When multiple conditions yield similar probabilities, diagnostic frequencies from historical datasets may be used to rank those conditions by prevalence to improve triage relevance and clinical prioritization. Severity level classifications may be determined by criteria including mortality rates, hospitalization frequency, time-sensitivity of treatment, and potential for permanent injury. The final output 112 may include probabilities 506 of the set of probabilities generated by the trained probabilistic classifier. The probabilities may represent the likelihood that the user has or will experience the condition 502. The probabilities 506 may be represented as percentages between zero and one hundred. Increased percentages correspond with greater likelihoods that the user is experiencing the condition, symptom, or disease, and lower percentages correspond with lower likelihoods that the user is experiencing the condition, symptom, or disease. The final output 112 may also include suggested actions 508 for the user to take. The suggested actions 508 may be determined based on the severity level 504 and the probability 506 of the condition 502. For example, if the severity level 504 is severe and the probability 506 is high, then the suggested actions 508 may include instructions for the user to go to the emergency room or to call an ambulance. If the severity level 504 is high but the probability 506 is low, the suggested actions may include scheduling an appointment to get a diagnostic test, such as an X-ray, completed within the next few days. If the severity level 504 is low and the probability 506 is high, the recommendation may include seeking out over-the-counter remedies. The final output ranking may alternatively be generated using ranking algorithms that learn to order conditions based on past diagnostic data, or multi-criteria decision analysis frameworks that combine probability scores, severity assessments, and user-specific risk factors through weighted aggregation functions.

In some embodiments, the final output 112 is compiled into a report in a digital or electronic format, such as a PDF or HTML format, that is sent to the user. The report may include the conditions 502, the severity levels 504, the probabilities 506 and the suggested actions 508. The report may also include confidence scores and relevant personal health history. In other embodiments, the pre-screening platform 102 includes an LLM that is programmed to generate Subjective, Objective, Assessment, and Plan (SOAP) notes. In these embodiments, the LLM used to generate SOAP notes may be a fine-tuned version of a transformer-based model, such as BERT or GPT, that has been trained on a dataset of de-identified clinical notes (e.g., clinical notes scrubbed of a patient's identifying data) and annotated SOAP-format records. The system may provide inputs to the model including structured data (e.g., symptom vectors, history, demographic information) and unstructured user input captured through the interface. The model may be trained to classify and organize relevant information into Subjective (patient-reported symptoms), Objective (vital signs, wearable data), Assessment (inferred likely conditions), and Plan (recommendations or triage actions) categories. In some embodiments, the model training uses a supervised learning technique with labeled clinical notes and reinforcement learning with expert-in-the-loop feedback (e.g., a medical practitioner provides the oversight in the supervised learning technique).

In some embodiments, the user interface may include an AI-driven avatar capable of text-to-speech (TTS) output. The TTS avatar may deliver the contents of the final output 112 through real-time verbal feedback, enhancing accessibility and user engagement. By synchronizing speech with animated visuals, the system helps clarify recommended next steps and relevant risk factors, particularly for users who prefer auditory guidance or benefit from multimodal interaction. The avatar system employing the TTS avatar may use commercially available platforms such as Amica AI, or a custom implementation built using open-source TTS models (e.g., FastSpeech2 or Tacotron 2) and facial animation models (e.g., Wav2Lip or SadTalker). The TTS model may generate natural-sounding speech from structured output, while the animation model synchronizes facial movements with speech and adjusts expressions based on semantic cues (e.g., urgency or severity of diagnosis). The TTS avatar may include both text-based output from the Bayesian inference model and annotated metadata (such as severity tags or confidence levels), as inputs, allowing the TTS avatar to adjust tone and emphasis in real time. In some embodiments, modules that recognize emotion or control speech patterns may be used to enhance expressiveness or align with user selections.

In some embodiments, the pre-screening system 100 includes a local user profile database that may be an additional input into the pre-screening platform 102. Rather than relying on a single static intake, the local user profile database refines each new evaluation using prior user input 104 and historical feedback. For example, the pre-screening system 100 may periodically update demographic data and/or medical history data used to generate the final output 112. In some cases, the pre-screening system 100 may update the demographic data and/or medical history data each time user input 104 is received, or each time the pre-screening system 100 processes the user input 104. In some embodiments, the local user profile database operates as a locally stored record that is updated after each user interaction. The user profile may be stored in a local user profile database on the mobile device. The system may encrypt the local user profile database using industry-standard encryption and store it in platform-specific secure storage. Data encryption and privacy protection measures for the locally stored user profile database may include Advanced Encryption Standard 256-bit (AES-256) encryption at rest, secure enclave storage on supported devices, and access controls requiring user authentication. In embodiments where at least a portion of the pre-screening system 100 operates in the cloud, the local user profile database may be off-device, and may be referenced each time that user input 104 is processed. The local user profile database may not directly retrain the global model in real time but instead may serve as personalized input for future inferences. Each time a user submits new user input 104, the system appends the user input 104 to the local user profile database and reuses the user input 104 to refine subsequent outputs, such as the final output 112. This allows the trained probabilistic classifier and the one or more embedding models to incorporate personalized historical context-such as prior symptoms, diagnosis likelihoods, and prior recommendations-without requiring full model retraining. In this way, the model achieves patient-specific accuracy improvements over time through adaptive input conditioning rather than continuous parameter updates. In some embodiments, the local user profile database may update predetermined embeddings or model parameters based on user feedback or interaction history through periodic batch updates or incremental learning techniques.

Some embodiments may incorporate an on-device AI model that performs the operations of the pre-screening system 100 offline when internet connectivity is unavailable. This on-device AI model can protect user privacy by storing sensitive information locally. In at least some embodiments, the on-device AI model may be a smaller “student” version as compared to a robust “teacher” model trained in the cloud to perform the operations of the pre-screening system 100. When a connection is established, the student model may synchronize with the cloud system for further refinement, and the system may transfer updates that include changes rather than the full model, model parameter averaging, or selective knowledge transfer focused on user-specific refinements.

In some embodiments, implementers may deploy the system within telemedicine platforms to provide automated triage before virtual consultations, enabling healthcare providers to prioritize patient queues based on symptom severity and probability assessments. The system may be integrated into electronic health record systems to augment clinical decision support by providing probabilistic condition assessments that clinicians can reference during patient encounters. The system may operate within insurance claim processing environments to evaluate symptom-condition relationships for coverage determination or to identify cases warranting additional medical review. The system may be deployed in occupational health settings to screen employees for work-related conditions, assess fitness for duty, or monitor health trends across workforce populations.

In some embodiments, one or more elements of the systems, methods, or computer-readable media described herein may be integrated into pharmacy management systems to evaluate patient-reported symptoms against medication profiles for drug interaction screening or identifying unwanted effects from medications. The system may operate within emergency dispatch systems to assist call center operators in triaging incoming calls and determining appropriate response levels based on caller-reported symptoms. The system may be deployed in clinical trial recruitment platforms to match patient symptom profiles and medical histories against trial eligibility criteria using semantic embedding comparisons. The system may be integrated into wellness and fitness applications to correlate user-reported symptoms with activity data, sleep patterns, and physiological metrics from connected devices. The system may operate within school health management systems to screen student-reported symptoms for communicable disease patterns and support decisions regarding attendance or isolation protocols.

FIG. 6 illustrates a flowchart for a routine 600 for healthcare pre-screening using machine learning, according to some embodiments. The routine 600 may be performed by the pre-screening system 100 described with reference to FIG. 1 and FIG. 2, and may be executed by the pre-screening platform 102 using the one or more trained embedding models and the trained probabilistic classifier. The routine 600 depicts a sequence of operations that transform user-provided symptom and health information into a ranked list of candidate conditions with associated probability scores.

The routine 600 begins at a block 602, where the pre-screening system 100 receives first data representing one or more symptoms experienced by a user and second data representing one or more of a medical history of the user, demographic information, or a health metric corresponding to the user. The first data 106 may include descriptions of symptoms such as pain, fatigue, discomfort, or other health concerns expressed by the user in natural language through the user interface. The second data 108 may include information about current medications, family medical history, prior hospitalizations, prior diagnoses, age, ethnicity, gender, environmental conditions, or other factors relevant to the user's health profile. In some embodiments, the block 602 may include prompting the user through the user interface to answer questions about symptoms, medical history, demographics, or health metrics. The first data and the second data may be received via text input, voice-to-text conversion, uploaded files, or other input mechanisms supported by the user interface.

The routine 600 proceeds to a block 604, where the pre-screening system 100 generates, using one or more trained embedding models, output embeddings by processing the first data and the second data. As described with reference to FIG. 2, the one or more trained embedding models may classify the output embeddings 202 into different categories such as symptom vectors, history vectors, and demographic vectors. The one or more trained embedding models may include Word2Vec models, FastText models, BERT models, GPT models, modified large language models, or compact embedding models such as DistilBERT, ALBERT, or TinyBERT configured for on-device execution. In some embodiments, multiple embedding models may be employed, with a routing mechanism directing different types of input data to specialized embedding models pre-trained for specific data categories.

The routine 600 continues to a block 606, where the pre-screening system 100 generates a user profile by comparing the output embeddings to one or more predetermined embeddings, such as by using a vector similarity function. The vector similarity function may include cosine similarity, dot product similarity, Euclidean distance, Jaccard similarity, Hamming distance, or learned metric functions implemented with Siamese networks or triplet networks. The user profile 204 may include a list of symptoms the user is likely experiencing based on the comparison, along with medical history factors, demographic information, or health metrics that matched predetermined embeddings. As described with reference to FIG. 3, the user profile 204 may include elements 302 representing matched symptoms and factors, corresponding descriptions 304, and health data identifiers such as ICDCM codes 306.

The routine 600 proceeds to a block 608, where the pre-screening system 100 generates a set of probabilities for candidate conditions by evaluating the user profile against one or more categories of medical information using a trained probabilistic classifier. The one or more categories of medical information may include wearable and biometric data, medical history records, demographic data, community health trends, and integrated symptom-health risk probability data, as described with reference to the other data 110 in FIG. 1. The trained probabilistic classifier may calculate probability estimates according to a relationship wherein a posterior probability of a candidate condition given symptoms and medical history is proportional to a product of a likelihood of the symptoms given the candidate condition and medical history and a prior probability of the candidate condition given the medical history. The trained probabilistic classifier may include a Bayesian inference model, Markov chain Monte Carlo sampling methods, variational inference approximations, or ensemble methods combining multiple probabilistic classifiers. The set of probabilities may be used to generate the dataset 206 containing candidate conditions with associated diagnostic codes 402, descriptions 404, and data associated with condition 406.

The routine 600 concludes at a block 610, where the pre-screening system 100 provides, via a user interface, candidate conditions. Each candidate condition is associated with a probability score from the set of probabilities. The probability 506 may represent the likelihood that the user has or will experience each condition 502, expressed as a percentage between zero and one hundred. In some embodiments, each candidate condition may also be associated with a severity level. The severity level may be determined by referencing stored severity mappings associated with health data identifiers, or dynamically calculated based on historical case outcomes, population-level mortality and morbidity data, and the user's individual risk profile. The final output 112 may also include suggested actions for the user based on the severity level and probability of each condition, such as instructions to seek emergency care, schedule an appointment, or monitor symptoms. In some embodiments, the final output 112 may be compiled into a report in a digital format, delivered through an AI-driven avatar with text-to-speech output, or formatted as SOAP notes for sharing with healthcare providers.

FIG. 7 illustrates a diagrammatic representation of a machine in the example form of a computing device 700 within which a set of instructions, for causing the machine to perform any one or more of the methodologies discussed herein, including the pre-screening operations, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while a single machine is illustrated, the term “machine” shall also be taken to include any collection of machines (e.g., computers) that individually or jointly execute a set (or multiple sets) of instructions to perform any one or more of the methodologies discussed herein.

The example computing device 700 includes a processing device 702, a main memory 704 (e.g., read-only memory (ROM), flash memory, DRAM such as synchronous DRAM (SDRAM), etc.), a static memory 706 (e.g., flash memory, static random access memory (SRAM), etc.), and a secondary memory (e.g., a data storage device 716), which communicate with each other via a bus 708.

Processing device 702 represents one or more general-purpose processors such as a microprocessor, central processing unit, or the like. More particularly, the processing device 702 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 702 may also be one or more special-purpose processing devices such as an application-specific integrated circuit (ASIC), a field-programmable gate array (FPGA), a digital signal processor (DSP), network processor, or the like. Processing device 702 is configured to execute the processing logic (e.g., instructions 724) for performing the pre-screening operations and steps discussed herein, including receiving user input, generating embeddings, evaluating user profiles, and providing final outputs.

The computing device 700 may further include a network interface device 720 for communicating with a network 728. The network interface device 720 enables the computing device 700 to communicate with the pre-screening platform, medical databases, and healthcare data sources over the network 728. This network connectivity allows the computing device 700 to access external datasets, synchronize user profile data, and communicate with healthcare systems. The computing device 700 also may include a video display 710 (e.g., a liquid crystal display (LCD) or a cathode ray tube (CRT)), an alpha-numeric input device 712 (e.g., a keyboard), a cursor control device 714 (e.g., a mouse), and a signal generation device 718 (e.g., a speaker).

The data storage device 716 may include a machine-readable storage medium (or more specifically a non-transitory computer-readable storage medium) 722 on which is stored one or more sets of instructions 724 embodying any one or more of the methodologies or functions described herein, such as instructions for an application 715. Application 715 may correspond to a pre-screening application that executes one or more operations and/or steps discussed herein, including embedding generation, user profile creation, Bayesian inference, and output generation operations. A non-transitory storage medium refers to a storage medium other than a carrier wave. The instructions 724 may also reside, completely or at least partially, within the main memory 704 and/or within the processing device 702 during execution thereof by the computing device 700, the main memory 704 and the processing device 702 also constituting computer-readable storage media.

The computer-readable medium 722 may also be used to store embedding models, user profile data, medical datasets, and configuration data used in connection with the pre-screening operations described herein. The computer-readable medium 722 may also store a software library containing methods for the application 715. While the computer-readable medium 722 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

As may be clear from the foregoing disclosure, embodiments of the present disclosure provide many benefits in comparison to conventional options. Embodiments of the present disclosure provide a healthcare pre-screening system that uses machine learning to assess patient symptoms and provide personalized health risk evaluations. The system receives data representing symptoms experienced by a user along with data representing the user's medical history, demographic information, or health metrics. Using one or more trained embedding models, the system generates output embeddings that capture the semantic meanings of reported user symptoms, eliminating the need for exact keyword matching. This allows users to describe their symptoms using different keywords or phrases than are used in medical libraries and still be matched to corresponding symptoms. The system generates a user profile by comparing the output embeddings to predetermined embeddings representing unique medical codes from symptom libraries such as ICD, DSM-5, CPT, or HCPCS. A trained probabilistic classifier, such as a Bayesian inference model, then compares the user profile to multiple datasets including wearable and biometric data, medical history records, demographic data, community health trends, and integrated symptom-health risk probability data to generate probability sets for candidate conditions. The final output provides users with a ranked list of conditions they may be experiencing, each with associated probability scores, severity levels, and recommended actions. This approach enables more accurate symptom matching through semantic understanding rather than exact keyword matching, incorporates personalized medical history and demographic data for better assessment, and provides quantified health risks in a user-friendly format to support informed decision-making. Compact embedding model architectures enable efficient on-device execution on mobile platforms, supporting user privacy by storing sensitive health information locally and providing responsive operation in connectivity-limited settings.

The foregoing description has presented illustrative, non-limiting examples and embodiments to facilitate understanding of the disclosed systems and methods for healthcare pre-screening and symptom assessment. Example implementations, architectures, data flows, parameters, and component arrangements have been provided for clarity and may be combined, omitted, substituted, or modified unless a particular feature is expressly stated as specified in a given context.

Throughout the foregoing Detailed Description, the following terminology and conventions have been used: The term “configured to” describes hardware, software, firmware, or combinations thereof arranged, programmed, or otherwise provisioned to perform a stated function. It refers to capability resulting from structure and/or programming, not mere intended use. The term “coupled” indicates direct or indirect interaction, association, or communication (for example, electrical, logical, data, network, message-passing, or shared-memory coupling), with or without intervening elements. Wired and wireless connections are included unless specified otherwise. The terms “about” or “approximately” indicate a value within normal engineering, measurement, or numerical tolerance for the context. Unless otherwise specified, “about” may encompass ±10% of the stated value or a tolerance appropriate to the resolution or precision stated. Numeric ranges are inclusive of endpoints and include all subranges and intermediate values. Where a range is recited with “about,” the approximation applies to endpoints and intermediate values unless otherwise indicated. Terms such as “optional,” “optionally,” and “may” indicate that a feature, step, or parameter can be present or absent, enabled or disabled, without implying that alternatives are excluded. Optional features can be used individually or in any combination.

Reference numerals and figure conventions have been applied as follows: The same reference numeral refers to the same or functionally equivalent element across figures. Suffixes (such as 102a, 102b) or primes (102′) may denote variants, instances, or related elements. Drawings are schematic and not necessarily to scale unless expressly indicated. Relative sizes, layer thicknesses, and spatial relationships may be exaggerated for clarity. Block diagrams depict functionality and/or logical groupings; a single block may represent multiple components or functions, and multiple blocks may be implemented within a single component. Flowchart steps may be performed in different orders, in parallel, iteratively, and/or with steps added or omitted, unless a particular order is expressly specified.

Units and coordinate systems have been used as follows: International System of Units (SI) units are used unless stated otherwise. Computing quantities may use standard units, such as bits, bytes, kibibytes (KiB), mebibytes (MiB), or gibibytes (GiB); computational performance may be expressed as floating-point operations per second (FLOP/s); time is expressed in seconds(s) or derivatives; frequencies are in hertz (Hz). Embedding vector dimensions and entity tracking data volumes may be expressed in bytes or kilobytes as appropriate for database storage contexts. Coordinate frames for diagrams are logical unless noted; references to “upstream/downstream,” “input/output,” or “producer/consumer” indicate dataflow directionality, not physical orientation.

Cross-references to figures have been provided for illustrative context (for example, “FIG. 1”). Figure numbers do not imply priority, sequence, or exclusivity. Components shown in different figures can be combined or substituted as technically appropriate.

These conventions have been applied throughout the Detailed Description, including descriptions of symptom embedding, user profile generation, Bayesian inference, probability estimation, condition assessment, and output generation, and to their variants and equivalents.

A number of implementations have been described. Nevertheless, it will be understood that various modifications may be made without departing from the spirit and scope of the disclosure. Accordingly, other implementations are within the scope of the following claims.

What is claimed is:

1. A system comprising:
at least one processing device; and
memory storing instructions that, when executed by the at least one processing device, cause the system to perform operations comprising:
receiving first data representing one or more symptoms experienced by a user and second data representing one or more of a medical history of the user, demographic information, or a health metric corresponding to the user;
generating, using one or more trained embedding models, output embeddings by processing the first data and the second data, wherein the output embeddings capture semantic relationships within the first data and the second data;
generating a user profile by comparing the output embeddings to one or more predetermined embeddings;
generating a set of probabilities for candidate conditions by evaluating the user profile against one or more categories of medical information using a trained probabilistic classifier; and
providing, via a user interface, one or more candidate conditions, wherein each of the one or more candidate conditions is associated with a probability score from the set of probabilities.

2. The system of claim 1, wherein the one or more categories of medical information comprise at least one of wearable data, biometric data, medical history records, demographic data, community health trends, or integrated symptom-health risk probability data.

3. The system of claim 1, wherein the trained probabilistic classifier calculates probability estimates according to a relationship wherein a posterior probability of a candidate condition given symptoms and medical history is proportional to a product of a likelihood of the symptoms given the candidate condition and medical history and a prior probability of the candidate condition given the medical history.

4. The system of claim 1, wherein the trained probabilistic classifier comprises at least one of a Bayesian inference model, a Markov chain Monte Carlo sampling model, a variational inference model, or an ensemble of multiple probabilistic classifiers.

5. The system of claim 1, wherein comparing the output embeddings to the one or more predetermined embeddings comprises using one or more of cosine similarity, dot product similarity, Euclidean distance, or Jaccard similarity, or using learned metric functions implemented with Siamese networks or triplet networks.

6. The system of claim 1, wherein the one or more trained embedding models comprise a compact embedding model configured for on-device execution on a mobile device platform, wherein the compact embedding model comprises a transformer-based architecture with fewer than 100 million parameters.

7. The system of claim 1, wherein each candidate condition is further associated with a severity level, and wherein the severity level is determined by at least one of: referencing stored severity mappings associated with a corresponding health data identifier; or dynamically calculating the severity level based on historical case outcomes and population-level data.

8. The system of claim 7, wherein the processing device is comprised in a mobile device, and wherein the system further comprises a local user profile database stored on the mobile device, wherein the local user profile database is encrypted and stored in platform-specific secure storage.

9. The system of claim 1, wherein providing the candidate conditions comprises providing a ranked list of candidate conditions ordered by probability score.

10. The system of claim 1, wherein the system employs a routing mechanism to direct different types of input data to specialized embedding models pre-trained for specific data categories based on at least one of input classification, keyword detection, or learned routing functions.

11. The system of claim 1, wherein the one or more trained embedding models comprise multilingual embedding models pre-trained on text corpora spanning multiple languages, enabling users to describe symptoms in multiple languages while maintaining accurate semantic matching.

12. The system of claim 1, wherein when the output embeddings do not meet a similarity threshold with any of the one or more predetermined embeddings, the system applies a fallback matching strategy using broader semantic categories.

13. The system of claim 1, wherein each of the one or more predetermined embeddings represents a health data identifier.

14. A method comprising:
receiving first data representing one or more symptoms experienced by a user and second data representing one or more of a medical history of the user, demographic information, or a health metric corresponding to the user;
generating, using one or more trained embedding models, output embeddings by processing the first data and the second data, wherein the output embeddings capture semantic relationships within the first data and the second data;
generating a user profile by comparing the output embeddings to one or more predetermined embeddings;
generating a set of probabilities for candidate conditions by evaluating the user profile against one or more categories of medical information using a trained probabilistic classifier; and
providing, via a user interface, one or more candidate conditions, wherein each of the one or more candidate conditions is associated with a probability score from the set of probabilities.

15. The method of claim 14, wherein the one or more predetermined embeddings are generated by embedding health data identifiers from a diagnostic code list comprising at least one of an International Classification of Diseases (ICD), a Diagnostic and Statistical Manual of Mental Disorders (DSM-5), a Current Procedural Terminology (CPT), or a Healthcare Common Procedure Coding System (HCPCS).

16. The method of claim 14, wherein when different categories of medical information provide contradictory evidence for a candidate condition, the trained probabilistic classifier resolves conflicting probability assessments through at least one of weighted averaging based on data source reliability, recency weighting, or hierarchical Bayesian aggregation.

17. The method of claim 14, wherein each candidate condition is further associated with a severity level, and wherein the severity level is determined by at least one of: referencing stored severity mappings associated with a health data identifier; or dynamically calculating the severity level based on historical case outcomes and population-level data.

18. A non-transitory computer-readable medium storing instructions that, when executed by one or more processors, cause the one or more processors to perform operations comprising:
receiving first data representing one or more symptoms experienced by a user and second data representing one or more of a medical history of the user, demographic information, or a health metric corresponding to the user;
generating, using one or more trained embedding models, output embeddings by processing the first data and the second data, wherein the output embeddings capture semantic relationships within the first data and the second data;
generating a user profile by comparing the output embeddings to one or more predetermined embeddings;
generating a set of probabilities for candidate conditions by evaluating the user profile against one or more categories of medical information using a trained probabilistic classifier; and
providing, via a user interface, one or more candidate conditions, wherein each of the one or more candidate conditions is associated with a probability score from the set of probabilities.

19. The non-transitory computer-readable medium of claim 18, wherein comparing the output embeddings to the one or more predetermined embeddings comprises using one or more of cosine similarity, dot product similarity, Euclidean distance, or Jaccard similarity.

20. The non-transitory computer-readable medium of claim 18, wherein each candidate condition is further associated with a severity level, and wherein the severity level is determined by at least one of: referencing stored severity mappings associated with a health data identifier; or dynamically calculating the severity level based on historical case outcomes and population-level data.
